(19) United States
(12) Patent Application Publication
(10) Pub. No.: US 2005/0013728 A1
(43) Pub. Date: Jan. 20, 2005

(54) Fragrance generation device

(75) Inventor: Yu-Fu Huang, Taipei (TW)

Correspondence Address: BROWDY AND NEIMARK, P.L.L.C.;624 NINTH STREET, NW, SUITE 300, WASHINGTON, DC, 20001-5303, US

(73) Assignee: FIRST INTERNATIONAL COMPUTER INC., Taipei (TW)

(21) Appl. No.: 10/620,622

(22) Filed: Jul. 17, 2003

(51) Int. Cl. A61L 9/00

(52) U.S. Cl. 422/4; 422/124

(57) ABSTRACT

A fragrance generation device for information products is described. A receiving space is formed at the bottom of a disk body to receive a fragrance sheet like a sponge so that a fragrance can be successfully emitted by the activity of a device such as a heat-radiating fan inside an information product like a computer, a PDA or an electric appliance. Foul smells like scorching can thus be masked when the information product operates. Moreover, the effect of aromatherapy can be accomplished simultaneously.

### BACKGROUND OF THE INVENTION

1. Field of the Invention

The present invention relates to a fragrance generation device for information products and, more particularly, to a fragrance generation device capable of letting an information product emit a fragrance when in use.

2. Description of Related Art

As shown in FIG. 1, the inside of an information product like a computer generally comprises a motherboard 10, a central processing unit (CPU) 11, a heat-radiating sheet 12 and a heat-radiating fan 13, all stacked together.

Along with continual progress of science and technology, operational speeds of information products become higher and higher. Heat generated therein increases therewith. The above components may easily generate fouls smell due to high temperatures after the power source is activated. Some people may even feel discomfort such as allergies due to the foul smells.

Accordingly, the above conventional information products like computers are inconvenience and have drawbacks in practical installation and use. The present invention aims to solve the problems in the prior art.

### SUMMARY OF THE INVENTION

The primary object of the present invention is to provide a fragrance generation device for information products, which lets an information product emit a fragrance when in use to accomplish the effect of aromatherapy.

To achieve the above object, the present invention provides a fragrance generation device for information products. A receiving space is formed at the bottom of a disk body to receive a fragrance sheet like a sponge so that a fragrance can be successfully generated by activity of, for example, a heat-radiating fan inside an information product such as, for example, a computer, a personal digital assistant (PDA) or an electric appliance. Foul smells like scorching can thus be masked when the information product operates.

### BRIEF DESCRIPTION OF THE DRAWINGS

The various objects and advantages of the present invention will be more readily understood from the following detailed description when read in conjunction with the appended drawing, in which:

FIG. 1 is a schematic, perspective view of the inside of a conventional information product;

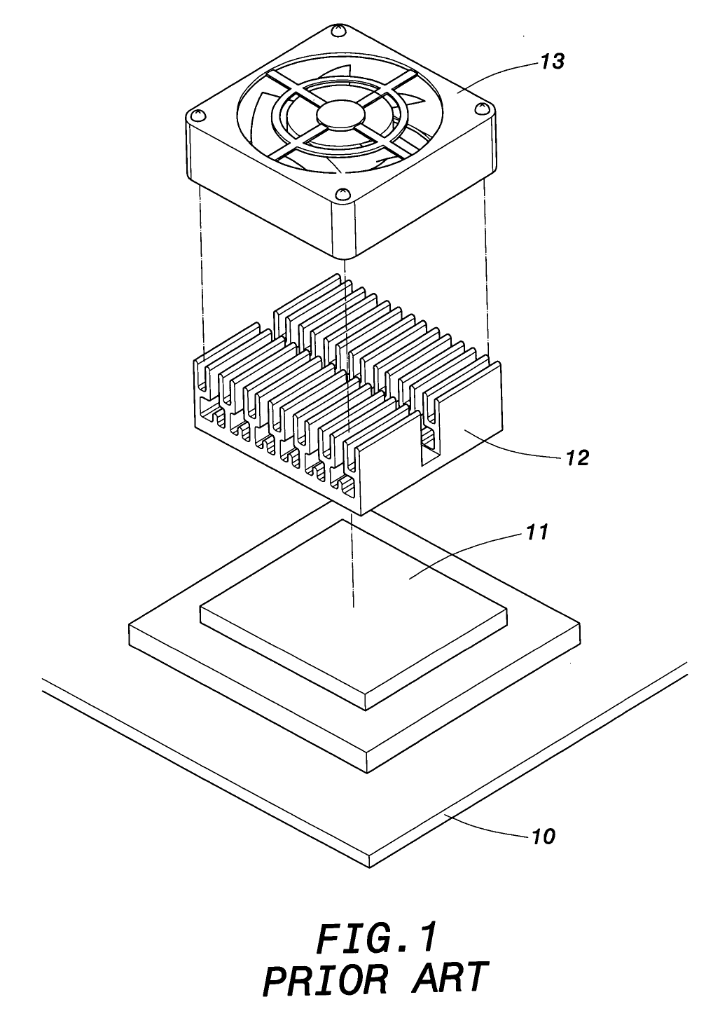

FIG. 2 is a schematic, exploded perspective view of the present invention;

FIG. 3 is a schematic, perspective assembly view of the present invention;

FIG. 4 is a schematic, cross-sectional view of the present invention;

FIG. 5 is another schematic, cross-sectional view of the present invention; and

FIG. 6 is a schematic, perspective assembly view according to another embodiment of the present invention.

### DESCRIPTION OF THE PREFERRED EMBODIMENTS

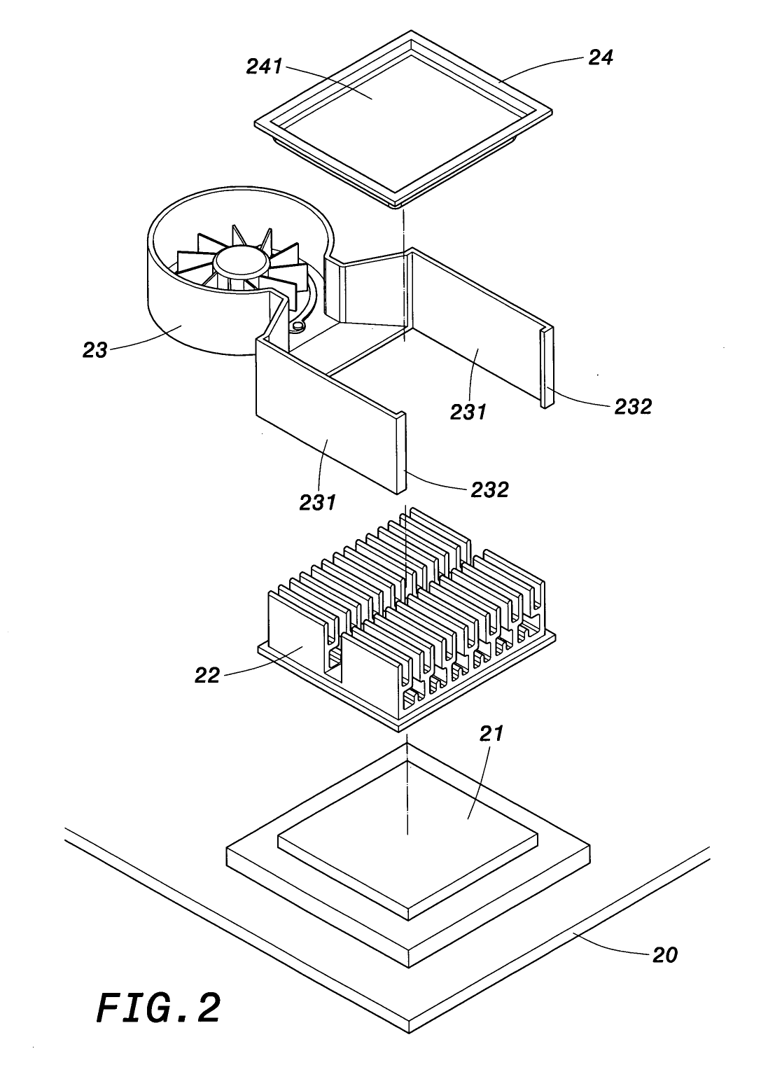

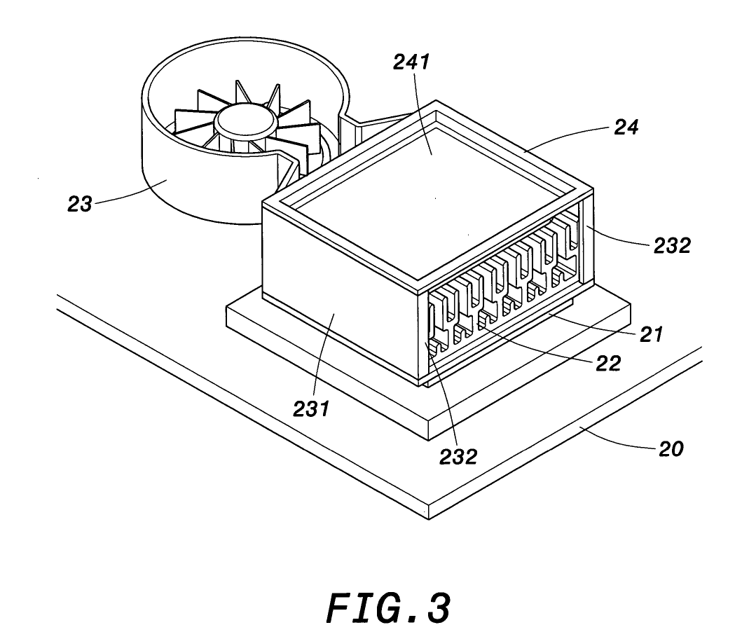

As shown in FIGS. 2 and 3, the present invention provides a fragrance generation device for information products, which lets an information product such as, for example, a computer, a PDA or an electric appliance emit a fragrance when in use. The fragrance generation device comprises a CPU 21, which gives out heat, a heat-radiating sheet 22 and a heat-radiating fan 23, all stacked together.

The CPU 21 is firmly inserted into a motherboard 20. The heat-radiating sheet 22 and the heat-radiating fan 23 are used to quickly radiate heat generated by the CPU 21 so that the information product can operate normally under permissible temperatures. As this structure is well know in the prior art, details thereof will not be further described below.

Reference is made to FIGS. 2 to 5. In the present invention, corresponding clamping arms 231 are formed on the heat-radiating fan 23 to clamp two sides of the heat-radiating sheet 22. One end of each of the clamping arms 231 forms a hook portion 232. The hook portions 232 are used to firmly hook the two sides of the heat-radiating sheet 22 to provide a firm connection, as shown in FIG. 3.

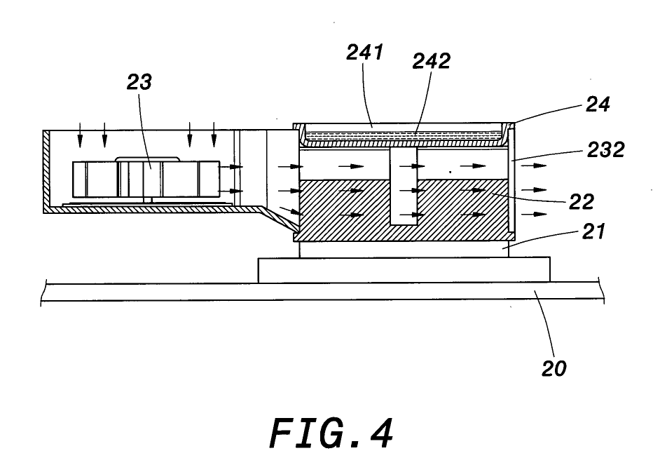

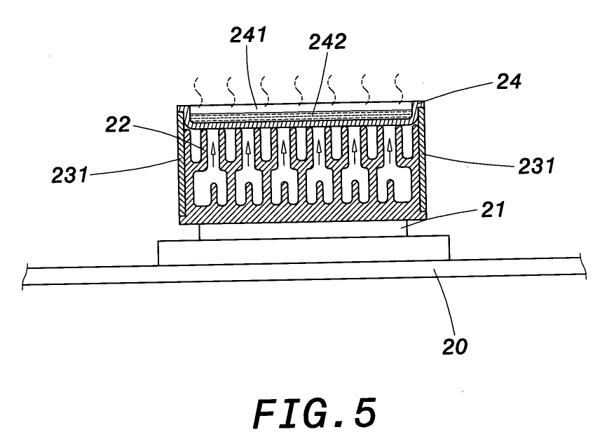

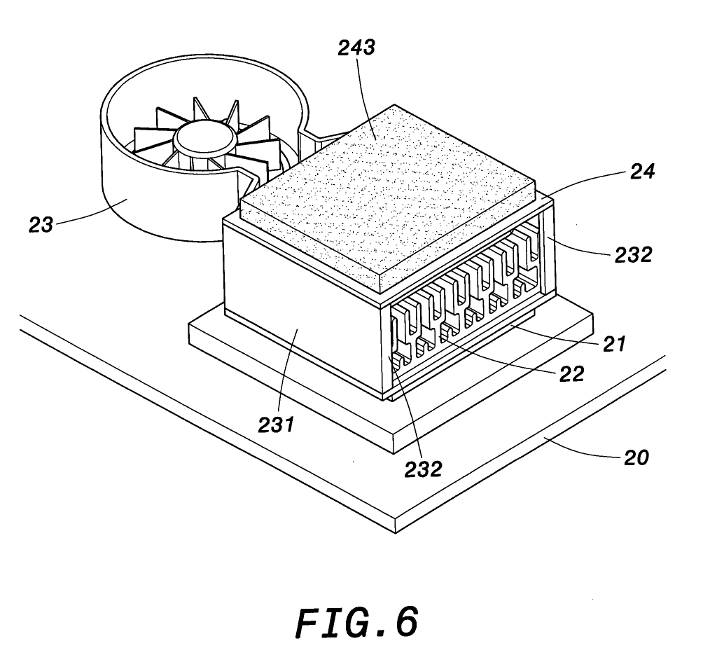

A disk body 24 can be directly or separately formed on the heat-radiating fan 23. The disk body 24 can be firmly clamped by the clamping arms 231. A receiving space 242 is formed at the bottom of the disk body 24. A fragrance sheet 242 can be received in the receiving space 241, as shown in FIG. 4. The fragrance sheet 242 can be an article having a high absorption capability like a sponge 243, as shown in FIG. 6. The fragrance sheet 242 can thus successfully emit a fragrance through the activity of the heat-radiating fan 23 to mask foul smells like scorching when the information product operates, as shown in FIG. 5.

To sum up, the fragrance generation device of the present invention has the following advantages:

1. Foul smells like scorch can be masked when an information product operates.

2. A user can select different fragrances according to his or her liking.

3. The effect of aromatherapy for stabilizing emotions can be accomplished to enhance work efficiency.

Although the present invention has been described with reference to the preferred embodiment thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

1. A fragrance generation device for information products, comprising:
a device giving out heat and disposed inside an information product;
a heat-radiating sheet stacked upon said device giving out heat;
a heat-radiating fan with a disk body disposed thereon, corresponding clamping arms being formed at two sides of said heat-radiating fan, said clamping arms being used to firmly clamp two sides of said heat-radiating sheet; and
a fragrance sheet received inside said disk body;
whereby a fragrance is emitted through activity of said heat-radiating fan inside said information product.

2. The fragrance generation device for information products as claimed in claim 1, wherein said information product is an electric article like a computer, a personal digital assistant or an electric appliance.

3. The fragrance generation device for information products as claimed in claim 1, wherein said fragrance sheet is a sponge.

4. The fragrance generation device for information products as claimed in claim 1, wherein said fragrance sheet is replaceable.
